(19) United States
(12) Patent Application Publication
(10) Pub. No.: US 2011/0250148 A1
(43) Pub. Date: Oct. 13, 2011

(54) COLD PROCESS WATER SOLUBLE WAX CONTAINING POWDERS

(75) Inventors: Juan R. Mateu, Oak Ridge, NJ (US); Adam Perle, Saddle River, NJ (US)

(73) Assignee: Jeen International, Fairfield, NJ (US)

(21) Appl. No.: 12/843,426

(22) Filed: Jul. 26, 2010

Related U.S. Application Data
(63) Continuation-in-part of application No. 12/832,547, filed on Jul. 8, 2010.
(60) Provisional application No. 61/347,664, filed on May 24, 2010.
(60) Provisional application No. 61/321,765, filed on Apr. 7, 2010.

(51) Int. Cl. A61K 8/92; A61Q 5/06; A61Q 5/12; A61Q 9/02; A61Q 1/12; A61Q 1/02; A61Q 19/04; A61Q 90/00; A61Q 5/02; A61Q 1/06

(52) U.S. Cl. 424/43; 424/64; 424/70.1; 424/59; 424/63

(57) ABSTRACT

Inventive subject matter described herein includes a powder comprising: a wax having a melt point within a range of 45 to 100 degrees Centigrade and sodium polyacrylate or salts of sodium polyacrylate or sodium polyacrylate and salts of sodium polyacrylate wherein the wax concentration falls within a range of 99.9% to 0.1% by weight and the sodium polyacrylate concentration falls within a range of 0.1% to 99.9% by weight, wherein the powder is effective for forming a cream or lotion or gel or coating when mixed with water at room temperature or cooler than room temperature.

### RELATED APPLICATIONS

This application is a continuation in part of and claims the benefit of priority under 35 U.S.C. §120 to U.S. patent application Ser. No. 12/832,547 filed on Jul. 8, 2010, which claims the priority benefit of U.S. Provisional Application Ser. No. 61/347,664 filed on May 24, 2010, and U.S. Provisional Application Ser. No. 61/321,765 filed on Apr. 7, 2010, the contents of which are incorporated herein by reference in their entirety.

### FIELD

Inventive subject matter described herein relates to powder embodiments that include one or more waxes and sodium polyacrylate or sodium alginate or a mixture of sodium polyacrylate and sodium alginate and to composition and method embodiments for lotions or creams or gels or coatings made with the powder embodiments, without an addition of heat.

### BACKGROUND

Emulsions, such as cosmetic emulsions, include mixtures of an oil phase that is dispersed in water. Other emulsions include an aqueous phase that is dispersed in oil; a siloxane phase that is dispersed in water; an aqueous phase that is dispersed in siloxane and an aqueous phase that is dispersed in oil to make an aqueous-oil phase that is dispersed in water.

Emulsions may include waxes, such as polyethylene, in the oil phase. Wax emulsions include formulated additives made with dispersed wax particles. Wax emulsions typically have particle sizes of less than one micron. Achieving stability of an emulsion, consistency and ease of use has been a challenge for at least the past 2000 years, since Roman women began adding a preparation of olive oil, water, beeswax and rose petals to their skin.

### SUMMARY

One embodiment described herein includes a powder comprising: a wax having a melt point within a range of 45 to 100 degrees Centigrade and sodium polyacrylate or salts of sodium polyacrylate or sodium polyacrylate and salts of sodium polyacrylate wherein the wax concentration falls within a range of 99% to 1% by weight and the sodium polyacrylate concentration falls within a range of 1% to 99% by weight, wherein the powder is effective for forming a cream or lotion or gel or coating when mixed with an aqueous medium without an addition of heat. For some embodiments, the temperature is room temperature or cooler than room temperature.

Inventive subject matter also includes a method of making a lotion or cream or gel or coating. The method includes mixing a powder with an aqueous medium at room temperature or a temperature cooler than room temperature to make the lotion or cream or gel or coating. The powder includes a wax having a melt point within a range of 45 to 100 degrees Centigrade and sodium polyacrylate or salts of sodium polyacrylate or sodium polyacrylate and salts of sodium polyacrylate wherein the wax concentration falls within a range of 99% to 1% by weight and the sodium polyacrylate concentration falls within a range of 1% to 99% by weight of the powder.

Inventive subject matter also includes a method of making a lotion or cream or gel or coating. The method includes mixing a powder with an aqueous medium without an addition of heat to make the lotion or cream or gel or coating. The powder includes a wax having a melt point within a range of 45 to 100 degrees Centigrade and sodium polyacrylate or salts of sodium polyacrylate or sodium polyacrylate and salts of sodium polyacrylate wherein the wax concentration falls within a range of 99% to 1% by weight and the sodium polyacrylate concentration falls within a range of 1% to 99% by weight of the powder.

Inventive subject matter also includes a method for making a powder. The method includes mixing one or more molten waxes having a melt point in a range of 45 to 100 degrees C. with sodium polyacrylate or one or more salts of sodium polyacrylate or both to form a mixture and spray-drying the mixture to form a powder.

Inventive subject matter includes one other method for making a powder. The method includes mixing one or more molten waxes having a melt point in a range of 45 to 100 degrees C. with sodium polyacrylate or one or more salts of sodium polyacrylate or both to form a mixture and prilling the mixture to form a powder.

### DETAILED DESCRIPTION

The following detailed description includes references to embodiments, which are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the inventive subject matter is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term “wax” refers to a linear non-polar carbon-based molecule. Exemplary waxes include natural waxes such as plant waxes and animal waxes. Plant waxes include candelilla wax, carnauba wax, sugar cane, rice bran wax, bayberry wax, sunflower wax, cocoanut butter, shea butter, and Japan wax. Animal waxes include beeswax, lanolin and whale wax. Petroleum waxes include microcrystalline wax and paraffin wax. Synthetic waxes include polyethylene wax and Fisher-Tropsch wax. Mineral waxes include montan, wax, ozokerite and ceresin. Waxes include fully hydrogenated waxes and partially hydrogenated waxes. Waxes usable herein include natural secretions of plants and animals; waxes made by purification from petroleum and waxes that are synthesized. Waxes include long chain alkanes, esters, polyesters of long chain primary alcohols and fatty acids. Waxes also include emulsifying waxes, such as glyceryl monostearate.

Specific waxes include polyethylene formulations manufactured by Jeen International, Corp. of Fairfield, N.J., including Jeenate 2H, Jeenate 3H, Jeenate 4H, Jeenate 5H and Jeenate 6H. As used herein, Jeenate 2H refers to a linear polyethylene having a melting point range of 54.0 to 62.0 degrees C. For some embodiments, the linear polyethylene has a melting point of 58 degrees C. Jeenate 3H is a linear polyethylene having a melting point range of 66.0 to 73.0 degrees C. Jeenate 4H is a linear polyethylene having a melting point range of 70 to 79.5 degrees C. Jeenate 5H is a linear polyethylene having a melting point range of 83 to 90 degrees C. Jeenate 6H is a linear polyethylene having a melting point range of 90 to 107 degrees C.

Other Jeen waxes that include a sodium polyacrylate described herein include Jeesperse CPW-1, CPW-2, CPW-3, obtained from Jeen International of Fairfield, N.J. Jeesperse CPW-2 and CW-3 include sodium polyacrylate and polyethylene. For some embodiments, the polyethylene includes one or more of Jeen's polyethylene 2H, polyethylene 3H, polyethylene 5H, CPW-B, beeswax and other waxes. Some powder embodiments herein include a single wax. Other embodiments include two or more waxes to make a wax blend. For other embodiments, shea butter and cocoa butter function as the wax embodiments described herein.

Sodium polyacrylate, also called, “acrylates copolymer” is a general term for copolymers of two or more monomers selected from a group that includes acrylic acid, methacrylic acid or one of their esters. Other copolymers of acrylic acid include ammonium VA/acrylates copolymer, sodium acrylates copolymer, ethylene/acrylic acid copolymer, ethylene/calcium acrylate copolymer, ethylene/magnesium acrylate copolymer, ethylene/sodium acrylate copolymer, ethylene/zinc acrylate copolymer, ethylene/acrylic acid/VA copolymer, acrylates/VP copolymer, acrylates/VA copolymer, steareth-10 allyl ether/acrylates copolymer, acrylates/dteareth-50 acrylate copolymer, acrylates/steareth-20 methacrylate copolymer, acrylates/ammonium methacrylate copolymer, styrene/acrylates copolymer, styrene/acrylates/ammonium methacrylate copolymer, ammonium styrene/acrylates copolymer, sodium styrene/acrylates copolymer, acrylates/hydroxyesters acrylates copolymer, methacryloyl ethyl betaine/acrylates copolymer, lauryl acrylate/VA copolymer, VA/butyl maleate/isobornyl acrylate copolymer, ethylene/methacrylate copolymer, vinyl caprolactam/VP/dimethylaminoethyl methacrylate copolymer, sodium acrylates/acrolein copolymer, VP/dimethylaminoethylmethacrylate copolymer, AMP-acrylates copolymer) are similar to acrylates copolymer in their function in cosmetics and personal care products. Polymers of acrylic acid and its salts (polyacrylic acid, ammonium polyacrylate, potassium aluminum polyacrylate, potassium polyacrylate, sodium polyacrylate) have similar properties. Other copolymers polymerized copolymerized with polyacrylates include Polyacrylamide and PVA, Sodium Polyacrylate Starch, Acrylamide/Sddoium Polyacrylate, Hydroxyethyl acrylate/Sodium Acrylodimethyl Taurate Copolymer, Acrylate copolymer, Acrylamide/Ammonium Acrylate Copolymer, Acrylates/Beheneth-25 Methacrylate/Steareth-30 Methacrylate Copolymer, Polyvinyl Alcohols (and derivatives or blends), PVP(derivatives and blends), Sodium/Carbomer, Carbomer, TEA-Carbomer, and Acrylates/C10-30 Alkyl Acrylate Crosspolymer. The term, “VA” as used herein refers to vinyl alcohol. The term “VP” as used herein refers to vinyl polymer.

Sodium polyacrylate generally has use as an absorbent, adhesive, binder, emulsion stabilizer, film former, emollient, suspending media, and a reagent for increasing viscosity.

Melting point of waxes employed in powder embodiments described herein span a melting point range of 45 to greater than 100 degrees C., depending upon the type of final product desired. Waxes may have any molecular weight, effective for the desired type of final product, and include mixtures of the specific waxes described herein, depending upon the type of final product desired.

It is believed that inventive embodiments disclosed herein include at least one molecule that is describable as “comb shaped.” Sodium polyacrylate is, for instance, comb shaped. By “comb-shaped” is meant that hydrocarbons make up the back of the “comb” while functional groups include the “teeth of the comb.” These molecular “comb shaped” embodiments are not rigid but are capable of flexing and twisting. The “teeth” of the “comb shaped” molecule are parallel to elements of a wax and, with a compatible ratio, correlate along the length of the wax. Thus, the “comb shaped” molecule and wax blend embodiments described herein induce polarity along the length of the wax. The “comb shaped” molecule and wax molecule blend embodiments described herein display greater polarity than emulsifiers and surfactants because the emulsifiers and surfactants have a smaller chain length and tend to have functionality (“teeth”) localized at one end of the molecule.

For some embodiments, it is believed that several emulsifier and/or surfactant molecules line up with a wax molecule to form a molecular aggregate having the same features of a single “comb-type” molecule, i.e. effective for forming a cream or lotion or coating or gel when mixed with an aqueous medium at room temperature or cooler than room temperature

One inventive embodiment described herein includes a powder that includes a wax having a melt point within a range of 45 to 100 degrees Centigrade and sodium polyacrylate or one or more salts of sodium polyacrylate or sodium polyacrylate and one or more salts of sodium polyacrylate wherein the wax concentration falls within a range of 99.9% to 0.1% by weight and the sodium polyacrylate concentration falls within a range of 0.1% to 99.9% by weight. The powder is effective for forming a cream or lotion or gel or coating when mixed with water at room temperature or cooler than room temperature. The powder is added to an aqueous medium without an addition of heat.

The inventive powder embodiments having a concentration range of 80% w/w wax to 20% w/w sodium polyacrylate have a particle diameter in a range of about 1 micron to 5 millimeters. For one embodiment, the diameter of the powder particles is one millimeter.

The structure of particles of inventive powder embodiments aid in the particles having solubility in aqueous media and non-aqueous media, without an addition of heat. The intermolecular forces responsible for melting and boiling points are also responsible for solubility. It is believed that the intermolecular forces of dipole-induced dipole interaction enable the particles described herein to solubilize at room temperature or cooler, without an addition of heat.

Powder embodiments described herein are made by blending molten wax with sodium polyacrylate or salt of sodium polyacrylate or both to make a mixture and then spray drying the mixture to form a powder embodiment. For some embodiments, the powder is then blended with a cold solution of a thickener such as sodium polyacrylate or sodium alginate or a mixture of both.

It is believed that the sodium polyacrylate water absorbent responds to water osmotically. Water is drawn into the center of the polyacrylate polymer and the sodium polyacrylate uncoils to form a web. Water or other aqueous media are held in place within the powder particle by this web of uncoiled sodium polyacrylate. When wax is melted and added to the sodium polyacrylate, it is believed that the resulting particle structure is lamellar, with layers of wax and polyacrylate enclosing water or other aqueous media. This lamellar structure imparts a property to the powder particles wherein they are soluble in water. For other embodiments, sodium alginate is used instead of sodium polyacrylate. For other embodiments, sodium alginate is used with sodium polyacrylate.

It has surprisingly been found that powder particles that include one or more waxes having a melt point within a range of 45 to 100 degrees C. or greater than 100 degrees C. for some embodiments and sodium polyacrylate or salts of sodium polyacrylate or both effectively form a lotion or cream or gel or coating when added to water at room temperature or a cooler temperature. The results are surprising because it has heretofore required an addition of heat to mix waxes in water to make a lotion or cream or gel or coating.

In one particular embodiment, the powder includes a first polyethylene component having a molecular weight of 200 and a melting point within a range of 54 to 60 degrees C., and a concentration of 80% w/w of the polyethylene formulation, the polyethylene formulation also including sodium polyacrylate in a concentration of 20% w/w of the first polyethylene formulation.

In another embodiment, the inventive powder includes a second polyethylene formulation that includes a polyethylene having a molecular weight of 300, a melting point of 66-73 degrees C., and a concentration of 80% w/w of the second polyethylene, the second polyethylene formulation also including sodium polyacrylate in a concentration of 20% w/w of the second polyethylene formulation. For some embodiments, the polyethylene formulation is available under the trademark, Jeesperse CPW-1, CPW-2, or CPW-3 obtained from Jeen International of Fairfield, N.J. For other embodiments, the powder includes a mixture of the first and second polyethylene formulations.

For other embodiments, the powder includes a first wax having a melting point of 54-60 degrees C., or a second wax having a melting point of 66-73 degrees C., or a blend of the two waxes. For other embodiments, the powder has a single wax with a melting point range of 45 to 100 degrees C. For other embodiments, the powder has two or more waxes having a melting point range of 45 to 100 degrees C. For other embodiments, the powder includes one or more waxes having a melting point of greater than 100 degrees C. These powder embodiments also include sodium polyacrylate or salts of sodium polyacrylate or sodium alginate or salts of sodium alginate in concentrations described herein. For some embodiments, the powder embodiments also include a particulate filler, such as mica or talc.

Other embodiments include silicone and employ one or more of Jeesilc DMBF,VHBF,DMLY, VHLV, which include bixvinyl dimethicone/dimethicone copolymer. The silicone-containing embodiments may also include waxes such as coconut butter, beeswax, and shea butter with polyacrylate.

For other embodiments, one or more gums and, for some embodiments, one or more associative thickeners in concentrations effective for imparting features of enhanced and rapid thickening to a desired degree are added to an aqueous medium, along with an inventive powder embodiment described herein, to make an aqueous base cream. For some embodiments, one or more gums are added to the wax and sodium polyacrylate to make powder particles. For other embodiments, the one or more associative thickeners are added to the wax and sodium polyacrylate to make powder particles. For other embodiments the one or more gums and one or more associative thickeners are added to the aqueous medium and the inventive powder embodiment is also added to the aqueous medium.

For other embodiments, an inventive powder embodiment is mixed with an oil phase or an emulsion. For some embodiments, the inventive powder embodiments include particles or flakes or beads or prills and are millimeter-sized. For some embodiments, the emulsion that includes the powder includes particles sized for spraying. For some embodiments, the particles are prepared with an aqueous medium to form a paste. The paste also does not require heating for use.

For some embodiments, the powder embodiments are mixable with an aqueous phase at room temperature or cooler to make the cream base. The powder embodiments do not require an addition of heat when blended with an aqueous medium. The step of mixing the powder with an aqueous phase at room temperature or cooler unexpectedly enhances hydration of the gum or gums in the thickener conditioner to a greater degree than would be expected using conventional methods. It is believed that this unexpected effect is due to increasing the surface area of the waxes as well as a deagglomeration and agglomeration prevention of the gum or gums and thickener particles by the waxes in the thickener conditioner. By maximizing the exposed surface area of the wax or waxes and gum or gums and associative thickener, the degree of hydration of the gum or gums and associative thickener is increased. The increase in hydration of the gum or gums and thickener enhances thickening of an aqueous cream. The powder embodiments also aid in forming an aqueous cream having a uniform consistency.

Additionally, water absorbing properties of the sodium polyacrylate component of the first and second polyethylene waxes, described above, and powder embodiments generally, allow for the development of unique and desirable textures in the aqueous cream, lotion, gel, coating and other products made with the aqueous cream at room temperature or cooler.

For other embodiments, inventive powder embodiments are mixable with an oil phase. For some embodiments, the oil phase and an inventive powder embodiment are added to an aqueous phase to produce an emulsion. For some embodiments, the oil phase includes one or more emulsifiers.

For other embodiments, an inventive powder embodiment is mixable with an emulsion after the emulsion has been formed. These embodiments produce enhanced and rapid thickening to a desired degree in an emulsion prepared using the inventive powder.

In addition to unexpected enhancement of hydration of the gum or gums in the thickening conditioner embodiments, the conditioner embodiments described herein reduce a greasy feel in final products that include the inventive powder embodiments. Sensorial quality of the finished product is enhanced. Final products also display a reduction in dusting.

One other benefit is that products made with the powder embodiments described herein do not reach their highest viscosity until the finishing stage of manufacturing. This feature allows for improved dispersion of actives, colors, and other ingredients in the final products.

While a first and second polyethylene are described herein, it is understood that powder embodiments can be prepared with a single wax or with three or more waxes falling within a melting point range of 45 to 100 degrees C.

Gums usable in or with the thickening powder embodiment include any of various substances that exude from certain plants. Gums include but are not limited to sweet gum, eucalyptus, balata, gutta balata, ammoniac, carrageenan, conoma, dragon's blood, euphorbium, olibanum, galbarnum, ghatti, agar, algin, alginic acid, cherry tree gum, guar gum, gum acacia, Bengal kino, gum kino. Mesquite gum, mucilage, karaya gum, gutta-percha, lacquer, opopanax, gum sangapenum, tragacanth, gum sangapenum, tragacanth, locust bean gum guggulu extract, furcellaran, alginate, tara gum, SGF gum, gum combretum, campestris and combinations of these gums.

Associative thickeners include cellulose, carbopol, bentone clays, carrageenan, pectin, and locust bean, salts of acrylates, Acrylic acids, and mixtures of these associative thickeners.

For some embodiments, mixing of the particles in the powder embodiment with an aqueous cream or lotion or gel or coating may be performed concurrently with mixing with an oil media. This concurrent addition and mixing is integratible into an existing manufacturing process.

Another embodiment includes making a hydrogel with a powder embodiment. One method embodiment includes mixing a powder that includes the first polyethylene wax formulation with sodium polyacrylate and second polyethylene wax formulation with sodium polyacrylate, described above, with water at room temperature or a cooler temperature. The product is a hydrogel.

For other powder embodiments, powders that include waxes, such as the first and second polyethylene formulations described above, both including sodium polyacrylate, are used to carry and condition one or more deodorizing materials such as sodium bicarbonate to make a deodorizing conditioner. In another embodiment, the powder embodiments described herein are blended with a natural deodorizer such as alum. In another embodiment, the powder embodiments are blended with the alum. The powder embodiments employed in these deodorizing conditioner embodiments include one or more waxes that have a melting point range that is effective for delivering alum or sodium bicarbonate or other deodorant onto skin and for imparting an improved sensorial effect, upon application to skin.

As discussed, a natural deodorizing powder is prepared by using alum and natural waxes and sodium polyacrylate or one or more salts of sodium polyacrylate or both, such as those described above. In another embodiment, the waxes are blended with the sodium polyacrylate or salts of sodium polyacrylate or both and a mixture of deodorants. For other powder embodiments, the powder includes the one or more waxes and sodium polyacrylate. This powder is then blended with alum or deodorant particles or blends of alum and deodorant particles.

The deodorizing particle embodiments deagglomerate or prevent agglomeration of particles of sodium bicarbonate or alum or mixtures of sodium bicarbonate and alum in a final deodorizer. By preventing agglomeration or deagglomerating, surface area of the deodorizing particles is increased. As a result, the strengths of deodorants in final products are increased over conventional deodorants having a comparable deodorizing concentration. Deodorizing powder embodiments may be blended with water to make a paste. Acceptable powder particle structure includes flakes beads, prills or other solid particles or pastes. For some embodiments, the deodorizing powder embodiments include particles sized for spraying.

For some embodiments, inventive powder embodiments are mixed with aluminum chlorhydrate.

Other powder embodiments include a single wax or blends of waxes, sodium polyacrylate and one or more fragrances and emollients. The concentration of the wax or waxes is effective for releasing the fragrances and emollients into a bath. For one embodiment, the fragrance-emollient system is used in a bath bead. The fragrance and emollient properties of a bath bead made with a powder embodiment are enhanced in terms of intensity and duration because the waxes promote dispersion of one or more fragrances and emollients in a bath bead. As discussed above, the powder inhibits agglomeration and deagglomerates fragrances, botanicals and emollients. As a result, the degree and duration of fragrance, botanical and emollient action in final products are increased over conventional beads.

Emollients include Glucamate LT, Guarsilk, MaizeThix, XanThix, Conditioning Thickener, Crothix Liquid, Crothix Pastilles, Glyceryl Stearate, Glycol Stearate, Polyquaternium 7.

Fragrances include single florals, floral bouquets, spicy fragrance, woodsy, oriental blends, and aldehydes and mixtures. Fragrances also include botanicals including algae, Angelica extract, anise seed extract, apricot extract, Arnica, artichoke, astragalus, basil, Bayberry, bearberry, bee pollen, Bergamot, birch bark, black cohosh, black current, bladderwrack burdock root, butchers broom, calendula, carrot flakes, diced carrot, cascara sagrada, celery seed, chamomile, chapparal, cherry bark, chrysanthemum, clove, coltsfoot, comfrey, cucumber, damiana, dandelion, dulse, Echinacea, elder, eleuthero, eucalyptus, fennel, fenugreek, gingko biloba, ginseng, goldenseal, gotu kola, grape, grapefruit, green cedar chips, guarana, gun powder tea, hawthorn, hibiscus, honeysuckle, hops, horsetail, Indian hemp root, Irish moss, jasmine, juniper berry, kelp, kola nut, lady's mantle, lavender, lemongrass, lemon peel, licorice root, linden, meadow sweet, milk thistle, mulberry, myrrh, neem, nettle, nori, oatmeal, orange peel, parsley leaf flakes, passion fower, patchouli, peppermint, pond lily root, pumpkin seed, radish seed, red cedar chips, red cedar shavings, red clover, red raspberry, red rosebuds, rosehips, rosemary, sage, skullcap, sea lettuce, sea palm sea protein St. John's Wort, strawberry, sunflower, sunflower seed, textured soy, thyme, tomato, valerian, vanilla, verbena, violet, wakame, watercress, white pond lily, white willow bark, wild cherry bark, wild yam, witch hazel, yarrow, and yohimbe, The fragrances and botanicals may be in a solid or liquid form or a mixture of solid and liquid.

Other embodiments include plastics preservative conditioner embodiments that include high melting waxes. For these embodiments, the high melting waxes are blended with acids such as citric, lactic, L-ascorbic and other organic acids or blends of organic acids to make a plastics preservative conditioner.

Another embodiment is a powder conditioner and includes a blend of waxes, such as described above and the sodium polyacrylate or salts of sodium polyacrylate or both, and fillers such as mica, talc, nylon, polyurethane, salts such as sodium bicarbonate, and other particles. Other fillers include clays, smectite, bentones, bentonites, and disteardimonium hectorite. The filler particles are pressed with the inventive powder particles. Pressing generates heat, due to pressure and friction. It is believed that the heat generation aids in making new powder textures and sensorial features compared to what are currently available.

Other embodiments include coatings for wax paper and other paper coatings. The inventive particles allow preparation of a coating without a need for heat addition.

Inventive conditioner embodiments described herein are usable in creams, lotions and foundations usable as sunscreens, organic and inorganic, stick products, such as antiperspirants and lipstick, after-sun bronzers, baby care products, baby oil, lip gloss, lip plumper, concealer, cosmetic foundation, moisturizer, shampoo, hair conditioner, styling cream, anti-aging cream, cold, cream, bar soap, body oil, body wash/cleanser, depillary, exfoliant scrub, hand cream, hormonal cream, liquid hand soap, mask, redness/rosacea cream, shaving cream, lip balm, tanning oil, wound treatment, after shave, anti-fungal treatment, baby wipes, bath soak, hand sanitizer, lubricant, spermicide, scar treatment, skin fading cream, lightener, toners, rash cream, around eye cream, baby lotion, baby soap, bandages, body firming lotion, body spray, cradle cap treatment, diaper cream, facial cleanser, feminine moisturizer, food moisturizer, hair removal waxes, hemorrhoid treatments, insect and pest repellants, makeup remover, cream for muscle and joint soreness, pain relief cream, shaving cream, sunless tanning, varicose and spider vein treatment, plastic preservatives, hydrogels, color cosmetics including mascara, eye shadow, highlighter and nail polish.

Products made with the embodiments described herein display long wear and transfer resistance. For some embodiments, products are strengthened by additional film formers.

Inventive subject matter also includes wax paper, coating paper, shoe polish, wood polish, auto polish, mold release, cheese coatings, waterproofing leather or fabric, encaustic paintings, crayons, and colored pencils, gelling peroxide solutions, hair bleach, stain removal carpets or any fabric, teeth whitening, wound care, antibacterials, detergents using sodium polyacrylate as a sequestering agent.

The following examples are presented herein to illustrate specific inventive embodiments and are not meant to limit the scope of invention embodiments.

### EXAMPLE 1

A wax powder that included polyethylene and sodium polyacrylate was prepared. The powder had a white appearance and a melt point within a range of 50.0 to 60.0 degrees Centigrade. The wax powder was soluble in aqueous media that was not heated. For some embodiments, the aqueous media was at room temperature or a temperature cooler than room temperature. The wax powder enabled the creation of new textures and product forms, including hydrogels, depending upon the aqueous media. Hydrogel embodiments were produced by adding the wax powder to aqueous media in a concentration of 5 to 10 percent by weight. Viscosity of a blend having 5% of the wax powder by weight was about 40,000 cp. Viscosity of a blend having 10% of the wax powder by weight was about 95,000 cp.

### EXAMPLE 2

Another wax powder included cetearyl alcohol, sodium polyacrylate, steareth-20, polysorbate 60, and PEG-150 stearate. The wax powder had a melting point within a range of 48.0 to 52.0 degrees Centigrade. The wax powder was mixable with cold water or other aqueous media to form stable emulsions. The wax powder was capable of thickening oil and aqueous phases.

Emulsions were made using the wax powder by adding an amount of wax powder within a range of 0.1 to 15% by weight. Water was added, without heat, and the mixture was mixed. The viscosity of a 5% by weight wax powder to water blend was about 55,000 cp. The viscosity of a 10% by weight wax powder to water blend was about 110,000 cp. Oil and water phases were then added.

The wax powder was usable in all emulsions, cosmetics, skin care products to improve stability, quickly hydrate, enhance textures, thicken, improve development of colors, and amplify feel of a product.

### EXAMPLE 3

One other product is a base cream. The base cream includes distilled water in a concentration of 80.4 percent by weight; the wax powder of Example 1 in a concentration of 4.0 percent by weight; PS-DMBF Aqua Base, manufactured by Jeen International of Fairfield, N.J.; demethicone in a concentration of 3.0 percent by weight; glycerine in a concentration of 3.3 percent by weight; isopropyl isostearate in a concentration of 3.3 percent by weight; and CAP-5, manufactured by Jeen International of Fairfield, N.J., in a concentration of 1.0 percent by weight. The PS-DMBF includes dimeticone, cetyl PEG/PPG-10 dimethicone, bis-vinyl dimethicone/dimethicone copolymer. The CAP-5 includes phenoxyethanol, caprylyl glycol, potassium sorbate, water, and hexylene glycol.

The base cream was prepared by mixing the water and wax powder of Example 1 to make a blend and adding remaining components to the blend.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

### EXAMPLE 4

Another embodiment included a mascara wax powder that included a wax, sodium polyacrylate, PVP, and iron oxide. Water was added to the powder in a concentration effective for providing a consistency desired in a mascara. Other additions included one or more of titanium oxide, iron oxide, Red Dye No. 7, calcium, barium, aluminum and other colorants. While a mascara was described, the wax powder is also usable to make other types of makeup including eye shadow and lipstick.

### EXAMPLE 5

A wax powder that included polyethylene and sodium polyacrylate was prepared. The powder had a white appearance and a melt point within a range of 50.0 to 60.0 degrees Centigrade. The wax powder was soluble in aqueous media that was not heated. For some embodiments, the aqueous media was at room temperature or a temperature cooler than room temperature. The wax powder enabled the creation of new textures and product forms, including hydroalcoholics, depending upon the aqueous media. Hydroalcoholic embodiments were produced by adding the wax powder to aqueous media in a concentration of 5 to 10 percent by weight to a blend having up to 30 percent of an alcohol by weight. In one embodiment, the alcohol is ethanol The resulting blend is a gel. Viscosity of a blend having 5% of the wax powder by weight was about 40,000 cp. Viscosity of a blend having 10% of the wax powder by weight was about 95,000 cp.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description

What is claimed is:

1. A powder comprising: a wax having a melt point of about 45 degrees Centigrade or greater and sodium polyacrylate or salts of sodium polyacrylate or sodium polyacrylate and salts of sodium polyacrylate wherein the wax concentration falls within a range of 99.9% to 0.1% by weight and the sodium polyacrylate concentration falls within a range of 0.1% to 99.9% by weight, wherein the powder is effective for forming a cream or lotion or coating or gel when mixed with an aqueous medium at room temperature or cooler than room temperature.

2. The powder of claim 1, further comprising a filler.

3. The powder of claim 1, further comprising one or more of an emulsifier, ester, oil, fragrance, active, colorant and silicone.

4. The powder of claim 1, wherein diameter of particles of the powder ranges from about 1 micron to 5 millimeters.

5. A cream or lotion or coating or gel or coating comprising the powder of claim 1 and water.

6. One or more of a cream, lotion, coating, gel, cosmetic foundation, sunscreen, organic and inorganic, stick product, lipstick, after-sun bronzer, baby care product, baby oil, lip gloss, lip plumper, concealer, moisturizer, shampoo, hair conditioner, styling cream, anti-aging cream, cold cream, bar soap, body oil, body wash/cleanser, depillary, exfoliant scrub, hand cream, hormonal cream, liquid hand soap, mask, redness/rosacea cream, shaving cream, lip balm, tanning oil, wound treatment, after shave, anti-fungal treatment, baby wipe, bath soak, hand sanitizer, lubricant, spermicide, scar treatment, skin fading cream, lightener, toners, rash cream, around eye cream, baby lotion, baby soap, bandages, body firming lotion, body spray, cradle cap treatment, diaper cream, facial cleanser, feminine moisturizer, food moisturizer, hair removal waxes, hemorrhoid treatments, insect and pest repellants, makeup remover, cream for muscle and joint soreness, pain relief cream, shaving cream, sunless tanning, varicose and spider vein treatment, plastic preservatives, color cosmetic, mascara, eye shadow made using the powder of claim 1.

7. One or more of wax paper, coating paper, shoe polish, wood polish, auto polish, mold release, cheese coatings, waterproofing leather or fabric, encaustic paintings, crayons, and colored pencils, gelling peroxide solutions, hair bleach, stain removal carpets or any fabric, teeth whitening, wound care, antibacterials, detergents using sodium polyacrylate as a sequestering agent made using the powder of claim 1.

8. The powder of claim 1, wherein the wax comprises one or more of candelilla wax, carnauba wax, sugar cane wax, rice bran wax, bayberry wax, and Japan wax, beeswax, lanolin, whale wax, microcrystalline wax, paraffin wax, polyethylene wax, Fisher-Tropsch wax, montan, wax, ozokerite and ceresin, fully hydrogenated waxes and partially hydrogenated waxes, natural wax secretions of plants and animals; waxes made by purification from petroleum and waxes that are synthesized; Waxes that include long chain alkanes, esters, polyesters of long chain primary alcohols and fatty acids; emulsifying waxes, glyceryl monostearate.

9. A method of making a lotion or cream or coating or gel, comprising: mixing the powder of claim 1 with an aqueous medium at room temperature or cooler for a time effective to make the lotion or cream or gel or coating.

10. A method of making a lotion or cream or coating or gel, comprising: mixing the powder of claim 1 with an aqueous medium without an addition of heat for a time effective to make the lotion or cream or gel or coating.

11. A method for making a powder, comprising:
mixing one or more molten waxes having a melt point of 45 degrees C. or greater with sodium polyacrylate or sodium alginate or one or more salts of sodium polyacrylate or sodium alginate or both to form a mixture; and
prilling or spray drying the mixture to form a powder.

12. The method of claim 11, further comprising mixing a powder with the one or more waxes and sodium polyacrylate or sodium alginate or one or more salts of sodium polyacrylate or sodium alginate to form the mixture.

13. The method of claim 11, further comprising mixing a powder with the one or more waxes and sodium polyacrylate or sodium alginate or one or more salts of sodium polyacrylate or sodium alginate to form the mixture

14. A powder comprising: a wax having a melt point of 45 degrees Centigrade or greater and sodium alginate or salts of sodium alginate or sodium alginate and salts of sodium alginate wherein the wax concentration falls within a range of 99.9% to 0.1% by weight and the sodium alginate concentration falls within a range of 0.1% to 99.9% by weight, wherein the powder is effective for forming a cream or lotion or coating or gel when mixed with an aqueous medium at room temperature or cooler than room temperature.

15. A powder comprising: a wax having a melt point of at least about 45 to 100 degrees Centigrade and sodium alginate or salts of sodium alginate or sodium alginate and salts of sodium alginate wherein the wax concentration falls within a range of 99.9% to 0.1% by weight and the sodium alginate concentration falls within a range of 0.1% to 99.9% by weight, wherein the powder is effective for forming a cream or lotion or coating or gel when mixed with an aqueous medium at room temperature or cooler than room temperature.

16. A method for making a powder, comprising:
mixing one or more molten waxes having a melt point in a range of at least about 45 degrees C. or greater with sodium polyacrylate or sodium alginate or one or more salts of sodium polyacrylate or sodium alginate or both to form a mixture; and prilling or spray drying the mixture to form a powder.

17. The method of claim 16, wherein a product made with the powder displays long wear and transfer resistance.

18. A powder comprising: a wax having a melt point at least about 45 degrees Centigrade and sodium alginate or salts of sodium alginate or sodium alginate and salts of sodium alginate wherein the wax concentration falls within a range of 99.9% to 0.1% by weight and the sodium alginate concentration falls within a range of 0.1% to 99.9% by weight, wherein the powder is effective for forming a cream or lotion or coating or gel when mixed with an aqueous medium at room temperature or cooler than room temperature.

19. A chemical complex comprising a wax and one or more molecules having functional groups effective for aligning with the wax, imparting a polarity along the length of the wax, wherein the chemical complex is effective for forming a cream or lotion or coating or gel when mixed with an aqueous medium at room temperature or cooler than room temperature.

20. A powder usable for making a mascara, comprising:
Wax, sodium polyacrylate; polyvinyl pyrrolidone; and iron oxide in concentrations effective for forming a gel upon addition of an aqueous medium, wherein the aqueous medium.

21. The powder of claim 20, further comprising one or more of titanium oxide, iron oxide, and colorant.
